(19) United States
(12) Patent Application Publication
(10) Pub. No.: US 2017/0009272 A1
(43) Pub. Date: Jan. 12, 2017

(54) IN-SILICO BASED TECHNIQUES IN THE IDENTIFICATION OF POTENT ß-GLUCORONIDASE INHIBITORS

(71) Applicants: Maria Yousuf, Karachi (PK); Zaheer-UL- Haq, Karachi (PK); Nimra Naveed Shaikh, Karachi (PK); Muhammad Iqbal Choudhary, Karachi (PK)

(72) Inventors: Maria Yousuf, Karachi (PK); Zaheer-UL- Haq, Karachi (PK); Nimra Naveed Shaikh, Karachi (PK); Muhammad Iqbal Choudhary, Karachi (PK)

(21) Appl. No.: 14/794,621

(22) Filed: Jul. 8, 2015

(51) Int. Cl. C12Q 1/34

(52) U.S. Cl. CPC C12Q 1/34; G01N 2333/924; G01N 2500/20; G01N 2500/04

(57) ABSTRACT

The invention relates to a method of identifying inhibitors against target receptor β-glucoronidase. Three compounds were found to be completely non-cytotoxic while, the remaining compounds showed moderate cytotoxicity.

### BACKGROUND OF THE INVENTION

β-Glucoronidase is an important glycosidase enzyme which catalyzes the hydrolysis of complex carbohydrates into simplest monomeric units. Its over-expression relates with, several type of cancers, including breast, colon and prostate cancer. To treat these disorders the available drug brands on the market are silymyrin, and its derivatives. Some other drugs such as Nialamide, Isocarboxazid, and Phenelzine have also been reported to inhibit GUS activity. However, camptothecin, a plant alkaloid, its derivatives hycamptin and camptosar have been reported to have been granted approval for clinical use, but cause severe side effects including cirrhosis of the liver. Therefore, to overcome these adverse effects, there is a strong need to search and identify lead candidates which possesses therapeutic potential against this target receptor.

### BRIEF SUMMARY OF THE INVENTION

In searching and identifying inhibitors, we conducted structure-based pharmacophore based virtual screening of an in-house database with large chemical space of a diverse class of compounds. We developed five structure-based-pharmacophore models. Three individual structure-based-pharmacophore models derived from the available PDB I.D, 3LPF, 3LPG, and 3K4D and two structure-based-shared feature and merged feature pharmacophore models derived by using Ligand Scout software 3.0 version.

Pharmacophore-based virtual screening of in-house data-base identified 1,249 hits, along with 66 reported inhibitors dataset these hit candidates (1,315) were subjected for docking studies, by using FRED 3.0.1 version which successfully docked the pharmacophore-based virtually screened hit candidates, FRED docked and score the candidates by using its scoring function Chemgauss-4, which was further rescored by using GOLD software of 5.1 version into Gold-score, Chem-score and ASP score.

Enrichment factor is an essential parameter to evaluate the efficiency of the docking and scoring comparative to a random selection of compounds, therefore enrichment factor was calculated for 5%, 10%, 15% and 20% for hit candidates of in-house data-base. For 5% of data-set enrichment factor of Chemgauss-4 scoring function was found to be as most efficient, while the rest of the 10%, 15% and 20% of data-base Chem-score scoring function of GOLD was found to be as efficient one. Therefore we selected the docked molecules of top ranked 5% enriched data-base and subjected for in-vitro screening. Out of 5% enrichment (68) compounds, 33 compounds were made available for in-vitro screening, Out of these, eleven (11) compounds showed potent inhibitory potential comparative to the standard (D-saccharic acid, 1,4-lactone). These compounds were also evaluated for cytotoxicity assay, and three compounds were found to be completely non-cytotoxic, however the remaining showed moderate cytotoxicity.

### BRIEF DESCRIPTION OF THE SEVERAL VIEWS OF THE DRAWING

FIG. 1 depicts a structure-based Pharmacophore derived from 3LPF with a Ligand Scout depicting the following Pharmacophore features Yellow spheres showed two hydrophobic methyl substituent. Yellow sphere showed one hydrophobic aromatic substituent. Green vector (arrow) showed the hydrogen bonding of NH with conserved water molecule HOH 680. Green vector arrow showed the H-bonding of hydroxyl OH with amino acid. Glu413A. Red vectors (arrows) showed the H-bonding of HOH 731, and HOH 733 with hydroxyl group OH. Gray spheres showed the excluded volume.

FIG. 2 depicts 2D Pharmacophore Model Structure-based pharmacophore (SBPs) derived from 3LPG

FIG. 3 depicts a 3D Pharmacophore Model consisting of Ligand Scout depicting the following pharmacophore features. Two yellow spheres showed the hydrophobic methyl benzene ring interacting with the amino acid Val473A, MSE447A, PHE448A. Yellow spheres showed the one hydrophobic aromatic ring and hydrophobic fluorine. Green vector (arrow) showed the H-bonding donor NH to the acceptor HOH 667. Red vector (arrow) showed the hydrogen acceptor of carbonyl group of aldehyde from donor HOH677. Gray spheres showed the excluded volume

FIG. 4 depicts 2D Pharmacophore Model, structure-based pharmacophore derived from 3K4D

FIG. 5 depicts 3D Pharmacophore Model for which the Ligand Scout depicting the following pharmacophore features. Two red vectors (arrows) showed of H-acceptors, one of carboxylate anion, and one of lactam carbonyl keto group. Three red-green vectors (arrows) showed the 3H donor/acceptor of three OH groups. One pointed sphere showed the negative ionizable area of carboxylate anion. Gray spheres showed the excluded volume.

FIG. 6 depicts 2D Pharmacophore Model Structure-based Shared and merged feature pharmacophore (SBPs) models: Shared and merged feature Structure-based pharmacophore models were also derived by using all three available individual PDB I.d 3LPF,3LPG and 3K4D.

FIG. 7 depicts 3D Pharmacophore Model. The model Depicting a five (5) element based shared feature pharmocophore model derived from PDB I.D, 3LPF, 3LPG and 3K4D. by using Ligand Scout, three (3) green vectors showing H-donors, five (5) red vectors showing H-acceptors, four (4) yellow spheres depicting lipophilicity with hydrophobic regions grey spheres showing the excluded volume, along with reference point set with a.a residues from active site contour.

FIG. 8 depicts 3D Pharmacophore Model. The model Depicting a merged feature pharmacophore derived model, from PDB I.d 3LPF, 3LPG and 3K4D. by using Ligand Scout, it comprises of the features, six(6) red vectors showing the H-acceptors, four (4) green vectors showing the H-donors, one red pointed sphere representing the negative ionizable area, six (6) spheres showing the hydrophobic region, grey spheres showing excluded volume along with reference point set with amino. acid residues from active site contour.

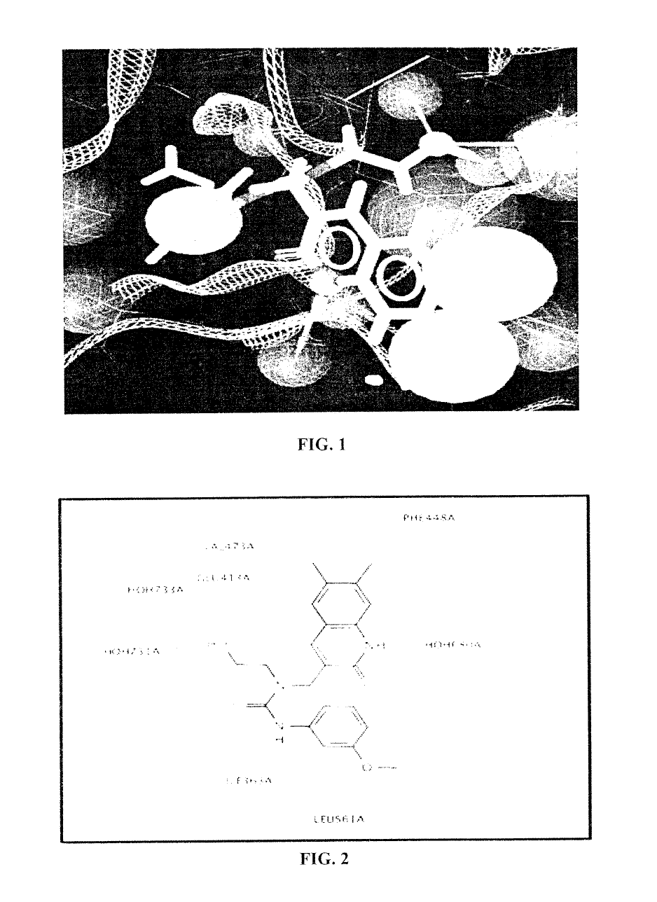

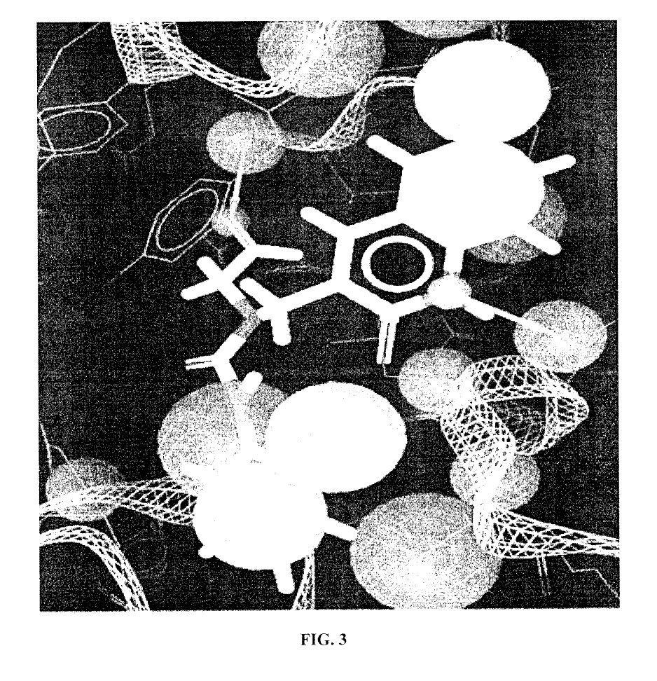

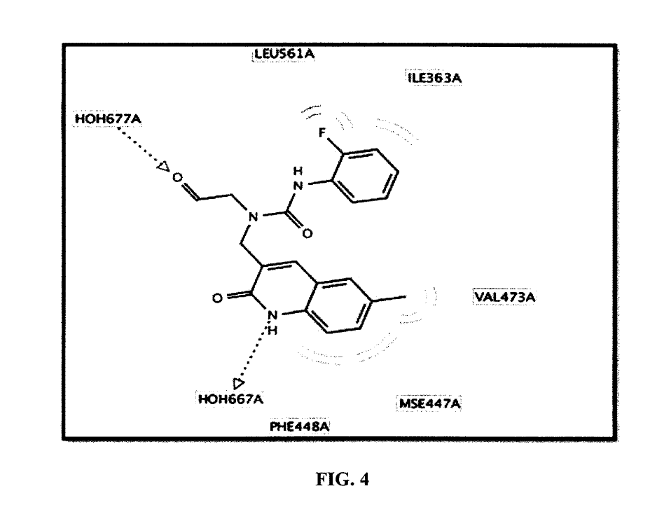

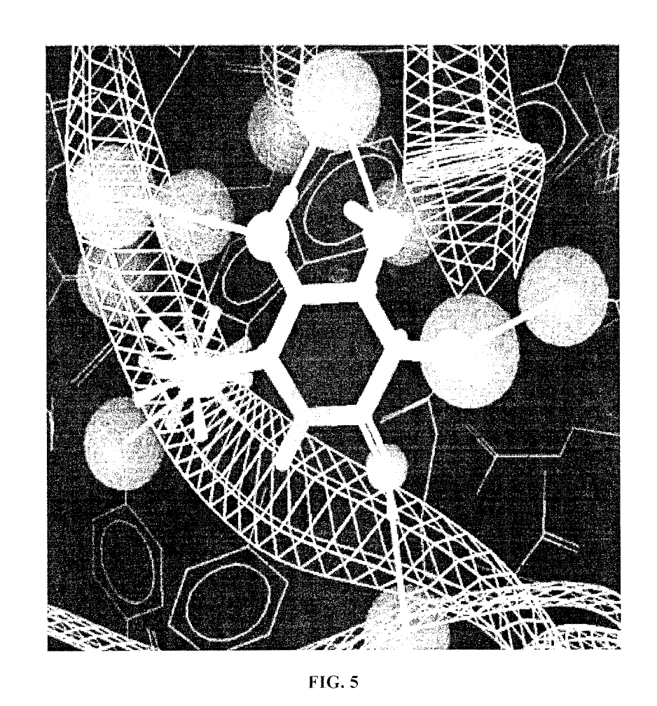

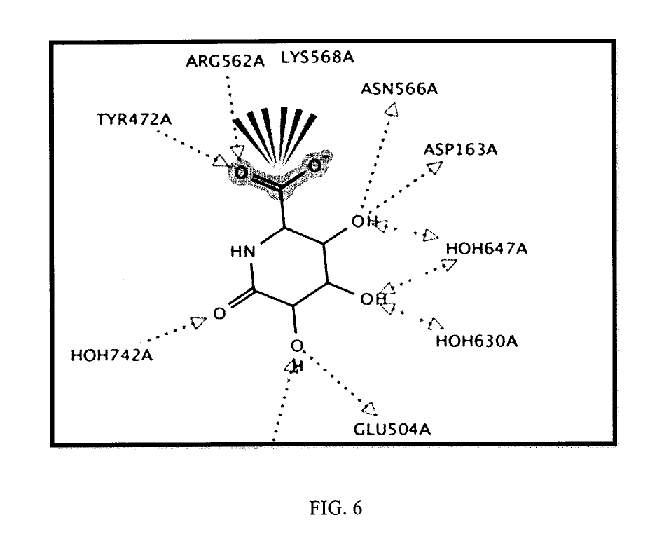

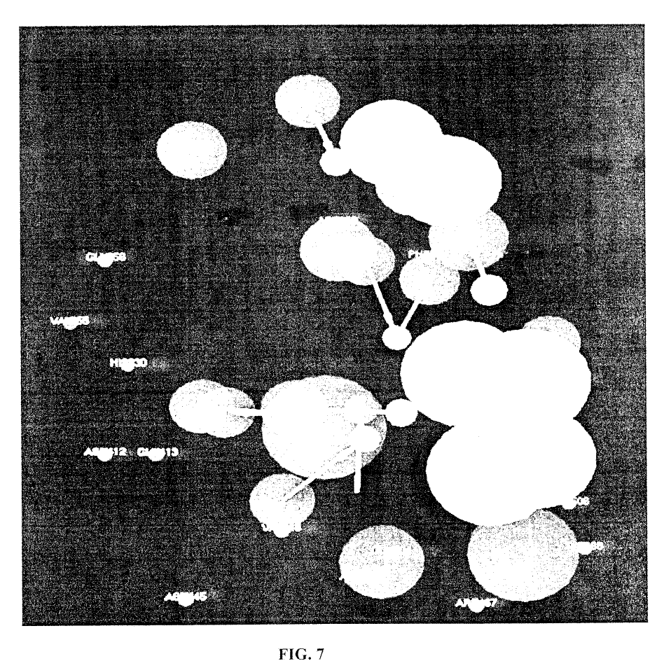

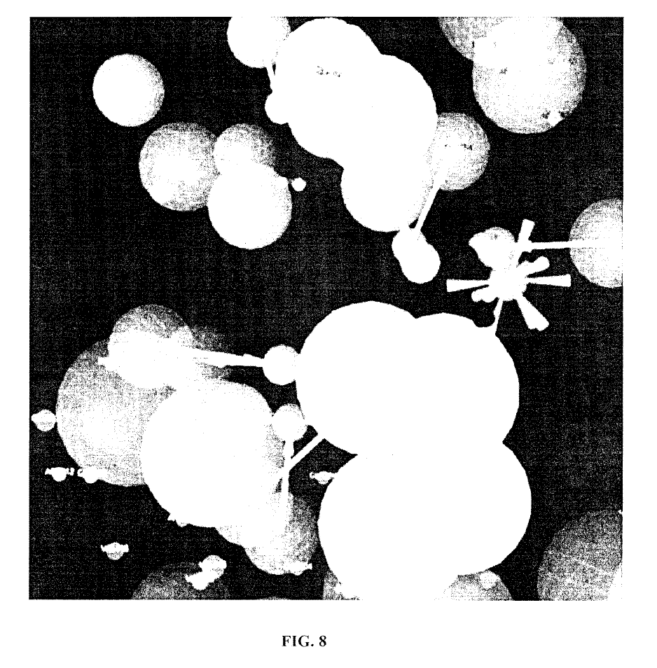

TABLE 1
2D docked poses of the potent inhibitors along with non-covalent interactions with the receptor β-Glucoronidase,
(Docked poses derived by software MOE, (Molecular Operating Environment).

| Compound No. | Ligand-receptor dock poses | Ligand-receptor interactions |
|---|---|---|
| 1 | 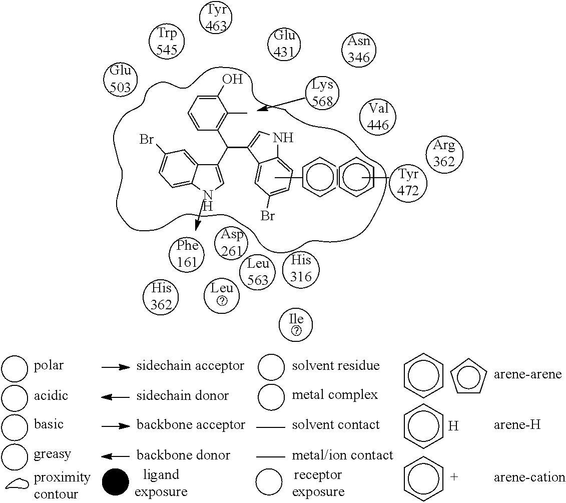 | NH of indol ring acting as backbone H-donor to Phe 161 for H-bonding, another phenyl ring of indol moiety showed arene-arene π-π stacking with Tyr 472 amino acid. 2-hydroxy substituted phenyl ring acts as acceptor from Lys 568 for H-bonding. |
| 2 | 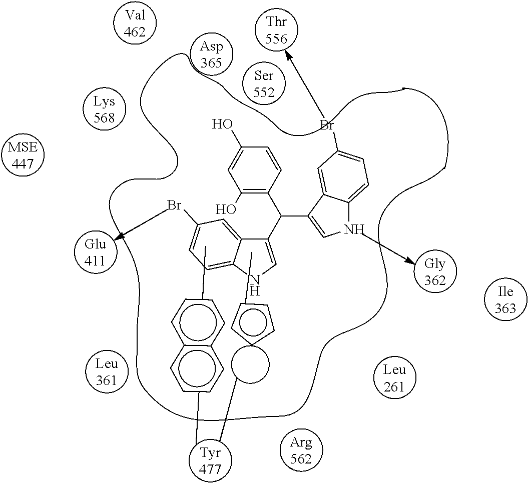 | 2-β-OH of pyranose substituent of coumarin moiety acting as OH- donor to Glu 503, 3-α-OH acts as- donor to Asp 161 for H-bonding, while 5-β-H acts as donor to Glu 413 for H-bonding |
| 3 | 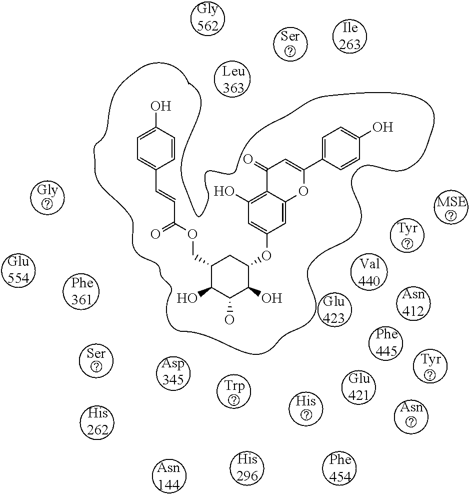 | 2-β-OH of pyranose substituent of coumarin moiety acting as OH- donor to Glu 503, 3-α-OH acts as donor to Asp 161 for H-bonding, while 5-β-OH acts as donor to Glu 413 for H-bonding |
| 4 | 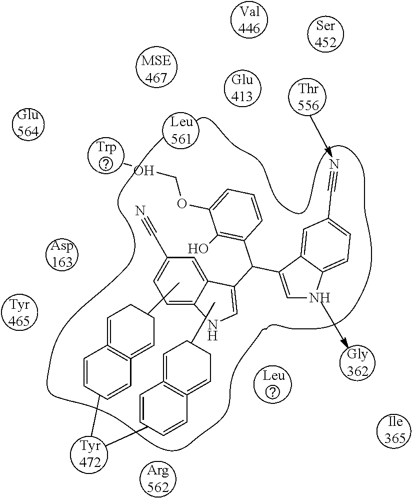 | Tyr 472 shows arene-arene π-π stacking interactions with indol ring. Trp 549 acts as arene-H donor to methoxy oxygen for H- bonding. Thr 556 acts as H- donor to the lone pair of azo- nitrogen |
| 5 | 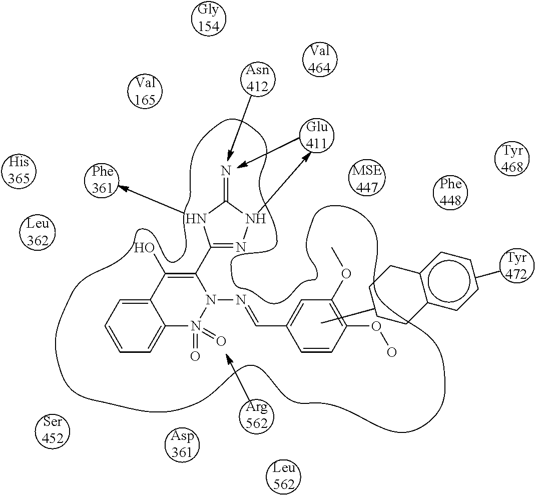 | S of thioimidazole (thione) acting as aceptor from Asn 412 and Glu 413. NH of thioimidazole acting as H-donor to Phe 161 and Glu 413. Arg 562 acting as H-donor to the sulphone group of compound. Tyr 472 showing arene arene π-π stacking with the aromatic ring of the compound |
| 6 | 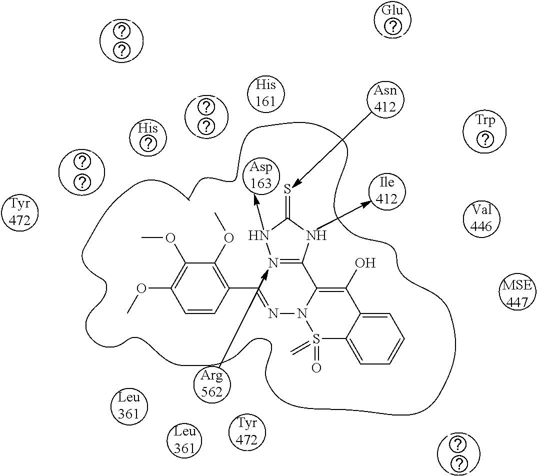 [SMILES: C=S1(=O)c2ccccc2C(O)=C(c2n[nH]c(=S)[nH]2)N1/N=C/c1ccc(OC)c(OC)c1OC] | One NH of cyclic thiourea acting as H-donor to Glu 413 for H- bonding, another NH of thiourea acting as H-donor to Asp 163 for H-bonding, Asn 412 acting as H-donor to the sulphur atom for H-bonding. Arg 562 acting as H-donor to the lone pair of N atom for H-bonding |
| 7 | 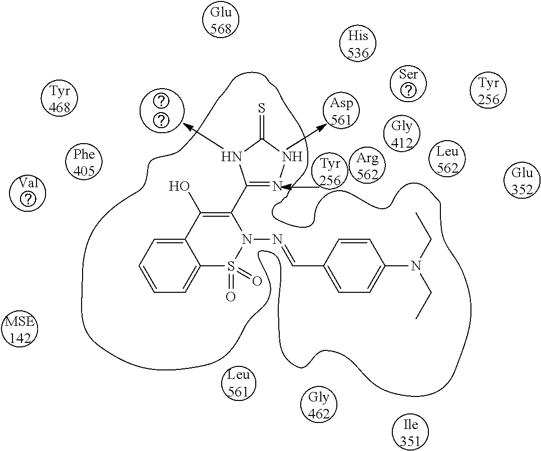 [SMILES: CCN(CC)c1ccc(/C=N/N2C(c3n[nH]c(=S)[nH]3)=C(O)c3ccccc3S2(=O)=O)cc1] | Thioimdazole (thione) moiety acting as H-donor for H-bonding to Asp 163 and Glu 413, Tyr 472 shows H-donor to the lone pair of N-atom for H-bonding |
| 8 | 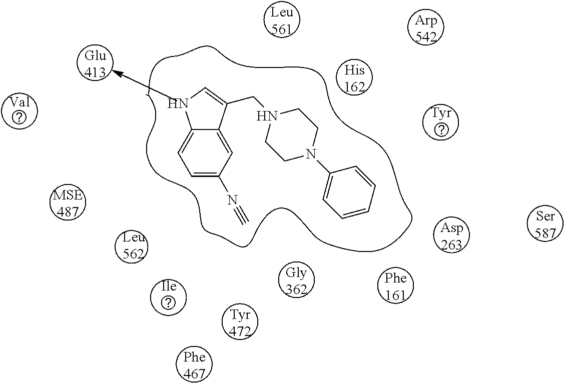 | NH of indol acting as H-donor to the Glu 413 for H-bonding |
| 9 | 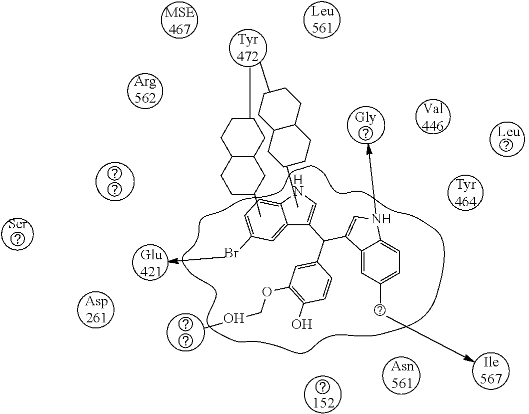 | Tyr 472 shows arene arene-π-π stacking interactions with the indol ring. NH acting as H- backgone donor to Gly 362. Br acting as lone pair donor to Thr556, another Br acting as lone pair donor to Glu 413, Try 549 showing arene-H interactions with methoxy substituent. |
| 10 | 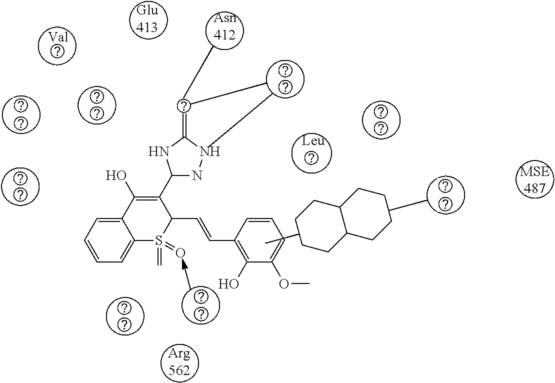 | One NH of thioimidazole (thione) acting as H-donor to Glu 413, while another NH acting as H-donor to Phe 161 for H-bonding Asn412 and Glue 413 acting as a H donor to the lone pair of sulphur atom and shows H-bonding. Tyr 472 shows arene arene π-π stacking interactions with hydoxy and methoxy substituted phenyl, Arg562 acting as H-donor to the oxygen atom of SO₂. |
| 11 | 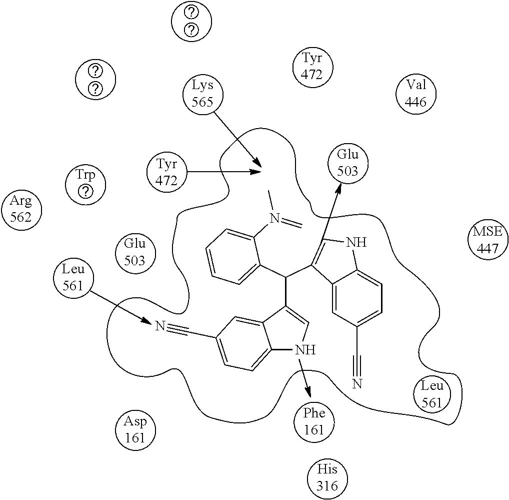 | Tyr 472, Lys 565 acts as H-donor to the oxygen atom or aromatic substituted NO₂ group for H- bonding, Leu 561 acts as H-donor to the lone pair of nitrogen atom of cyano group. NH of pyrol ring showing H-donor to the Phe 161. |

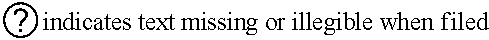

### DETAILED DESCRIPTION OF INVENTION

In the present application virtual screening hit results, (scaffold hopping) has been successfully performed, and we identified in top 5% enriched data-base, new classes of compound with potent biological activity against β-glucoronidase, which are not cytotoxic against 3T3 mouse fibroblast cell line. Therefore, these compounds will be used to evaluate the β-glucoronidase activity at the in-vivo level, and other further later steps of drug designing and discovery process.

Structure-based pharmacophore mapping was the keen step which took the ligand-receptor information and developed the model, which searched and identified the inhibitors in the large chemical space (8,262) compounds. Structure-based Pharmacophore (SBPs) model was derived from protein-ligand complexes which illustrate the potential interactions exist between ligand and protein. It is a useful tool for medicinal chemists to identify novel ligands which fulfill the pharmacophore requirements and have a high probability of being biologically active. This has been proven and validated from my structure-based pharmacophore mapping and virtual screening. We developed five structure-based-pharmacophore models, three individual structure-based-pharmacophore models derived from the available PDB I.D, 3LPF, 3LPG, and 3K4D and two structure-based shared feature and merged feature pharmacophore models were derived by using Ligand Scout software 3.0 version.

In the models, the most repeated, keen interactions are b/w Glu413, Tyr472 and Phe161 of the active site amino-acid residues with ligands, the similar interactions are also observed in our potent inhibitors. Therefore, an efficient virtual screening defined in terms of new scaffolds hopping (searching of structurally new and novel compounds). Low hit rates of interesting scaffolds are always preferable over high hit rates of already known scaffolds. Usually a series of compound becomes active against a targeted receptor, but here a scaffold hopping results due to structure-based Pharmacophore model. The details of structure-based Pharmacophores along with the interactions are as follows.

In-silico based theoretical step were used at first than experimentally evaluated the identified Hits, which is the rational approach towards drug designing and discovery process. Usually people use in-silico techniques after the experimental work.

During in-silico based screening we first used Lipiniski ROF based filters (Omega filter from Open eye), which filtered the unstable and toxic compounds from data-base, and selected those compounds which followed the drug ability criteria so that our compounds would show non-cytotoxicity.

These compounds were also used to evaluate the cytotoxicity against normal fibroblast 3T3 cell line of mouse. Three compounds were found to be completely non-cytotoxic while, the remaining compounds showed moderate cytotoxicity. The list of the potent inhibitors, compounds with activity data, are as follows:

TABLE 2
Bio-assay screening and cytotoxicity Results:

| | | | IC₅₀ (μM) of | |
|---|---|---|---|---|
| Compounds No. | % inhibition | IC₅₀ (μM) | cytotoxicity | |
| 1 | 97.8 | 1.20 ± 1.03 | 16.84 ± 0.997 | |
| 2 | 99.2 | 1.373 ± 0.64 | 10.512 ± 0.487 | |
| 3 | 96.9 | 4.50 ± 0.44 | >30 | |
| 4 | 62.1 | 8.5 ± 1.43 | 20.122 ± 0.584 | |
| 5 | 78.1 | 11.41 ± 0.04 | 13.783 ± 0.967 | |
| 6 | 81.0 | 11.8 ± 0.86 | 17.133 ± 1.411 | |
| 7 | 95.1 | 14.7 ± 1.88 | >30 | |
| 8 | 73.4 | 15.3 ± 2.30 | >30 | |
| 9 | 75.9 | 16.16 ± 0.76 | 9.536 ± 0.327 | |
| 10 | 93.4 | 16.65 ± 0.69 | 19.548 ± 1.074 | |
| 11 | 57.3 | 34.9 ± 0.21 | 9.564 ± 0.134 | |
| Standard | 89.4 | 45.45 ± 2.16 | St inhibitor | 0.2 |
| Inhibitor | | | cycloheximide | |
| D-sacharric 1,4- | | | | |
| lactone | | | | |

Following are the bio-assay protocol used to evaluate the biological activities of compounds against the enzyme β-glucoronidase and normal cell line of mouse fibroblast.

β-Glucuronidase inhibition assay protocol: Inhibitory activity of β-Glucuronidase was determined with the help of spectrophotometric method by measuring the absorbance at 405 nm of p-nitro phenol formed from the substrate (p-nitro phenyl-β-D-glucuronide, N1627-250 mg, Sigma Aldrich). The total reaction volume was 250 μL. The compound dissolved in DMSO (100%), which becomes 2% in the ultimate assay (250 μL) and the similar conditions were used for standard (D-saccharin acid 1,4-lactone, Sigma Aldrich). The reaction mixture contained 185 μL of 0.1 M acetate buffer, 5 μL of test compound solution, 10 μL of (1U) enzyme solution (G7396-25KU, Sigma Aldrich) was incubated at 37° C. for 30 min. The plates were read on a multiplate reader (SpectraMax plus 384) at 405 nm after the addition of 50 μL of 0.4 mM p-nitrophenyl-β-D-glucuronide. All assays were performed in triplicate. IC₅₀ Values were calculated by using EZ-Fit software (Perrella Scientific Inc., Amherst, Mass., U.S.A.). These values are the mean of three independent readings.

Cytotoxicity assay Protocol: Cytotoxic activity of compounds was evaluated in 96-well flat-bottomed micro plates by using the standard MTT (3-[4,5-dimethylthiazole-2-yl]-2,5-diphenyl-tetrazolium bromide) colorimetric assay (15). For this purpose, 3T3 (mouse fibroblast) cells were cultured in Dulbecco's Modified Eagle Medium, supplemented with 5% of fetal bovine serum (FBS), 100 IU/ml of penicillin and 100 μg/ml of streptomycin in 75 cm² flasks, and kept in 5% CO₂ incubator at 37° C. Exponentially growing cells were harvested, counted with haemocytometer and diluted with a particular medium. Cell culture with the concentration of 5×10⁴ cells/ml was prepared and introduced (100 μL/well) into 96-well plates.

After overnight incubation, medium was removed and 200 μL of fresh medium was added with different concentrations of compounds (1-30 μM). After 48 hrs, 200 ptL MTT (0.5 mg/ml) was added to each well and incubated further for 4 hrs. Subsequently, 100 μLof DMSO was added to each well. The extent of MTT reduction to formazan within cells was calculated by measuring the absorbance at 540 nm, using a micro plate reader (Spectra Max plus, Molecular Devices, Calif., USA). The cytotoxicity was recorded as concentration causing 50% growth inhibition (IC₅₀) for 3T3 cells. The percent inhibition was calculated by using the following formula

% inhibition=100−((mean of O.D of test compound−mean of O.D of negative control)/(mean of O.D of positive control−mean of O.D of negative control)*100).

The results (% inhibition) were processed by using Soft-Max Pro software (Molecular Device, USA).

1. (canceled)

2. (canceled)

3. A β-glucuronidase enzyme inhibitor selected from a group of compounds consisting of 3-(bis(5-bromo-1H-indol-3-ypmethyl)benzene-1,2-diol; (E)-((2,S,3R,4R,5S,6R)-3,4,5-trihydroxy-6-(5-hydroxy-2-(4-hydroxyphenyl)-4-oxo-4H-chromen-7-yloxy)tetrahydro-2H-pyran-2-yl)methyl 3-(4-hydroxyphenyl)acrylate; 5-(2-{[3,4-dimethoxyphenyl]methylidene)amino}-4-hydroxy-1,1-dioxo-2H-1,2-benzothiazine-3-yl)-2,4-dihydro-3H-1,2,4-triazole-3-thione; 5-(2-[{2,3,4-trimethoxyphenyl)methylidene]amino}-4-hydroxy-1,1-dioxido-2H-1,2-benzothiazine-3-yl)-2,4-dihydro-3H-1,2,4-triazole-3-thione; 5-(2-[{4-N-N′-dimethylaminophenyl)methylidene]amino}-4-hydroxy-1,1-dioxido-2H-1,2-benzothiazine-3-yl)-2,4-dihydro-3H-1-1,2,4-triazole-3-thione; 1-((5-(methyldyneammonio)-1H-indol-3-yl)-4-phenylpiperazin-1-ium; and 5-(4-Hydroxy-2-{[(E)-(2-hydroxy-3-methoxyphenyl)methylidene]amino}-1,1-dioxido-2H-1,2-benzothiazin-3-yl)-2,4-dihydro-3H-1,2,4-triazole-3-thione.

4. The β-glucuronidase enzyme inhibitor of claim 1, wherein said β-glucuronidase enzyme inhibitor is used to treat breast, colon and prostate cancer.
